(19) United States
(12) Patent Application Publication
(10) Pub. No.: US 2021/0193321 A1
(43) Pub. Date: Jun. 24, 2021

(54) COMPUTATIONAL SYSTEMS AND METHODS FOR DIAGNOSING AND TREATING MOOD DISORDERS

(71) Applicant: Kevin Rogers, Vancouver (CA)

(72) Inventor: Kevin Rogers, Vancouver (CA)

(21) Appl. No.: 17/198,163

(22) Filed: Mar. 10, 2021

Related U.S. Application Data
(63) Continuation-in-part of application No. PCT/US2020/049780, filed on Sep. 8, 2020.
(60) Provisional application No. 62/897,366, filed on Sep. 8, 2019.

(51) Int. Cl. G16H 50/20; G16H 20/60; G16H 20/70; G16H 10/20

(52) U.S. Cl. CPC G16H 50/20; G16H 10/20; G16H 20/70; G16H 20/60

(57) ABSTRACT

Systems, methods and devices for assessing and more efficiently treating mood disorders within a subject are provided, having the steps of: (a) conducting a lifestyle assessment; (b) conducting a biological assessment; and/or (c) conducting a behavioral assessment. All of (a), (b), and (c) can then be introduced to an AI engine capable of exceeding human diagnostic capabilities, and which over multiple iterations can produce therapeutic recommendations with greater preciseness for a given subject. All levels of assessment, lifestyle, biochemical, and behavioral may be conducted online and/or with computational systems.

### CROSS-REFERENCE TO RELATED APPLICATION

This patent application is a continuation-in part of International Application No. PCT/US2020/049780, filed Sep. 8, 2020, which application claims the benefit under 35 U.S.C. § 119(e) of U.S. Provisional Patent Application No. 62/897,366 filed Sep. 8, 2019; each application is incorporated herein by reference in its entirety for all purposes.

### FIELD OF THE INVENTION

The present invention relates generally to the field of diagnosing and treating mood disorders utilizing a variety of diagnostic tests and computational methods.

### BACKGROUND

According to the National Institute of Mental Health, around 40% of adults in the United States received some sort of treatment for mental dysfunction in 2017. Such disorders are a substantial burden to those afflicted, as well as to their friends, family and/or coworkers. Moreover, such disorders represent a significant detriment to the productivity of workers, as well as a burden to the health care system as a whole. Some estimates suggest that the cost to society is on the order of over $300 billion every year.

Mood disorders are generally diagnosed solely by patient disclosures to a physician or therapist. Such disclosures are however typically based solely upon a patient's subjective understanding of their condition and can be hampered by a myriad of issues making accurate diagnosis and treatment difficult, if not impossible.

The present invention solves a number of problems associated with current diagnostic methods for assessing and treating patients with mood disorders, and further provides other related advantages.

All of the subject matter discussed in the Background section is not necessarily prior art and should not be assumed to be prior art merely as a result of its discussion in the Background section. Along these lines, any recognition of problems in the prior art discussed in the Background section or associated with such subject matter should not be treated as prior art unless expressly stated to be prior art. Instead, the discussion of any subject matter in the Background section should be treated as part of the inventor's approach to the particular problem, which in and of itself may also be inventive.

### SUMMARY

Briefly stated, the invention relates to systems and methods for diagnosing and treating mood disorders. Within one aspect of the invention methods are provided for assessing a mood disorder within a subject, comprising the steps of: (a) conducting a lifestyle assessment; (b) conducting a biochemical assessment; and/or (c) conducting a behavioral assessment. Within certain embodiments of the invention the biochemical/biological assessment is a neurohormonal assessment which measures the presence, level or quantity of one or more of: serotonin, dopamine, GABA, Glutamate, PEA, norepinephrine, epinephrine, cortisol, DHEA, estradiol, progesterone, and testosterone. Within other embodiments of the invention the behavioral assessment determines the level of stress, anxiety and/or depression of the subject.

This Brief Summary has been provided to introduce certain concepts in a simplified form that are further described in detail below in the Detailed Description. Except where otherwise expressly stated, this Brief Summary is not intended to identify key or essential features of the claimed subject matter, nor is it intended to limit the scope of the claimed subject matter.

The details of one or more embodiments are set forth in the description below. The features illustrated or described in connection with one exemplary embodiment may be combined with the features of other embodiments. Thus, any of the various embodiments described herein can be combined to provide further embodiments. Aspects of the embodiments can be modified, if necessary, to employ concepts of the various patents, applications and publications as identified herein to provide yet further embodiments. Other features, objects and advantages will be apparent from the description, the drawings, and the claims.

### BRIEF DESCRIPTION OF THE DRAWINGS

Exemplary features of the present disclosure, its nature and various advantages will be apparent from the accompanying drawings and the following detailed description of various embodiments. Non-limiting and non-exhaustive embodiments are described with reference to the accompanying drawings, wherein like labels or reference numbers refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. One or more embodiments are described hereinafter with reference to the accompanying drawings in which:

FIG. 1 is a block diagram which illustrates an exemplary method of assessing and providing recommendations for mood disorders.

FIG. 2A is a block diagram which provides examples of queries for obtaining profile information on a subject.

FIG. 2B is a block diagram which provides queries for a lifestyle assessment.

FIG. 3 is a block diagram which illustrates an exemplary method of conducting biological assessments.

FIG. 4 is a block diagram which illustrates an exemplary method of conducting a behavioral assessment, and illustrative components thereof.

FIG. 5 is a block diagram which illustrates an exemplary method of conducting a professional assessment and/or an AI-driven assessment.

FIG. 6 is a block diagram which illustrates an exemplary method of providing therapeutic recommendations.

FIG. 7 illustrates one embodiment of message types and cadence of diagnosis and treatment.

FIG. 8 diagrammatically illustrates a computerized system for collecting and processing information.

FIG. 9 diagrammatically illustrates one embodiment of various phases of treatment and possible responses thereto;

FIG. 10 diagrammatically illustrates various different subjects' possible progression during the course of multiple phases of diagnosis and treatment.

FIG. 11 diagrammatically illustrates one embodiment of data evaluation.

FIG. 12 diagrammatically illustrates one embodiment of an automated support and advisory system.

FIG. 13 diagrammatically illustrates one embodiment of a recommendation system.

### DETAILED DESCRIPTION OF THE INVENTION

The present invention may be understood more readily by reference to the following detailed description of preferred embodiments of the invention included herein.

Prior to setting forth the invention in more detail however, it may be helpful to an understanding thereof to first set forth definitions of certain terms that are used hereinafter.

“Mood disorders”, as used herein refers to disorders wherein a subject's general emotional state or mood is distorted or inconsistent with their circumstances and interferes with their ability to function. Representative examples of mood disorders include: 1) acute or chronic stress, 2) anxiety, and 3) depression. Further examples of mood disorders can be found in the American Psychiatric Association. (2013). Diagnostic and statistical manual of mental disorder (5th ed.). Arlington, Va., American Psychiatric Association 2013 (hereinafter referred to as “DSM-V” which is incorporated by reference in its entirety). Anxiety and depression are listed in the DSM-V and have many variants that depend on severity and duration of experience. Stress related disorders do not, as such, have their own devoted section in the DSM-V with the exceptions of some acute or virulent stress reactions such as PTSD, which are listed in the anxiety disorders section.

“Neurotransmitters” refers to specific molecules that reside in and affect neuronal activity within the brain. “Neurohormones” refers to select adrenal hormones that cross the blood-brain barrier to affect brain function and behavior and that work within the body and brain to influence mood, energy levels, and an overall sense of wellbeing (or lack thereof). Representative examples of neurotransmitters and neurohormones include GABA; Phenylethylamine; Serotonin; Dopamine; Epinephrine; Norepinephrine; Cortisol; Histamine; Glycine and Glutamate. “Neurohormonal assessment” refers to the measurement and imbalances associated with neurotransmitters and neurohormones (as well as metabolites thereof).

In order to further an understanding of the various embodiments herein, the following sections are provided which describe various embodiments: A. Mood Disorders; B. Assessment of Biological Markers (including neurotransmitters and neurohormones); C. Behavioral and Lifestyle Assessments; and D. Devices, Systems and Methods for Assessing, Diagnosing, and Treating Mood Disorders.

### A. Mood Disorders

As noted above, the present invention provides methods for assessing and treating mood disorders. including for example (1) Depressive Disorders; (2) Anxiety Disorders; and (3) Trauma and Stressor Related Disorders (see generally DSM-V as cited above). Each of these mood disorders is discussed in more detail below.

1. Depressive Disorders

Depressive Disorders are characterized by the presence of sad, empty, and/or irritable moods, accompanied by somatic and/or cognitive changes that affect a subject's capacity to function. Representative examples of Depressive Disorders include: disruptive mood dysregulation disorder; major depressive disorder; persistent depressive disorder; premenstrual dysphoric disorder; substance/medication-induced depressive disorder; and other specified and unspecified depressive disorders.

2. Anxiety Disorders

Anxiety Disorders are characterized by excessive fear and anxiety, as well as related behavioral disturbances. Representative examples include: separation anxiety disorders; selective mutism; specific phobias; social anxiety disorders, panic disorders, agoraphobia, generalized anxiety disorders, anxiety disorders due to another medical condition; and other specified and unspecified anxiety disorders.

3. Trauma and Stressor Related Disorders

Trauma and Stressor Related Disorders are characterized by psychological distress due to exposure to a traumatic or stressful event. Representative examples include: reactive attachment disorders; disinhibited social engagement disorders; posttraumatic stress disorders; acute stress disorders; adjustment disorders; and other specified and unspecified trauma and stressor related disorders. While chronic stress is not represented in the DSM-V it is still recognized as a “mood disorder” and, in fact, determined, through abundant research, to be a key “precipitator” of both anxiety and depression—both from its effect on neuro-hormonal chemistry and its effect on mood disruption and cognitive and cerebral fatigue.

### B. Assessment of Biological Markers (Including Neurological Hormones)

As noted above, within at least one embodiment of the invention an assessment of biological markers is made, in order to assist in providing a therapeutic assessment of the subject. In the context of the present invention biological markers can include any of a number of different biological tests that can be run on a subject, including for example: vitamin (e.g., vitamins A, C and/or D) levels and mineral levels; markers of inflammation (e.g., C reactive protein); hormonal levels; metabolic markers; blood glucose; genetic markers; allergy tests; gut microbiome determinations and neurotransmitter/neurohormones. Within preferred embodiments of the invention the biological marker has been tied to one or more mood disorders.

Within a particularly preferred embodiment of the invention the subject can be tested for the level of a number of neurotransmitter/neurohormones that may influence a mood disorder. Representative examples are discussed below.

1. Gamma-Aminobutyric Acid or “GABA”

Gamma-Aminobutyric acid or “GABA”, is one of the major inhibitory neurotransmitters in the brain. It acts at inhibitory synapses in the brain by binding to specific transmembrane receptors (of which there are two classes: GABA_{A} and GABA_{B}. A large number of drugs have been developed either as allosteric modulators of GABA receptors, or to increase the amount of available GABA. These drugs typically have a relaxing, anti-anxiety effect on patients.

2. Serotonin

Serotonin is a monoamine neurotransmitter that sends signals from the enterochromaffin cells within the digestive system which secrete and connect to specific 5-HT receptors. Their secretion increases the circulation of the platelets and activation; where it proceeds to rouse the myenteric neurons and gastrointestinal motility. The serotonin that is left over is synthesized in the serotonergic neurons that exist within the central nervous system. Here, the serotonin plays a role with almost all human behaviors including mood, sleep, appetite, and sexuality.

3. Glutamate

Glutamate is, of the amino acid neurotransmitters, the most abundant. Glutamate is the neurotransmitter that helps to regulate brain development. Glutamate can also have a somewhat toxic effect as it essentially excites cells to death, in a process called excitotoxicity. High amounts of glutamate have been linked to many diseases such as Alzheimer's, and lathyrism. It attaches to the receptor NMDA, AMPA, kainite, and mGluR), and has even been linked to depression.

4. Epinephrine

Epinephrine (also known as adrenaline) is a monoamine neurotransmitter that plays an essential role in the fight or flight response. As a hormone epinephrine is connected to almost all of the body tissues. As an excitatory neurotransmitter/neurohormone epinephrine can influence such body reactions as increasing heart rate and respiratory rate in the lungs, can relax and contract smooth muscles, as well as stimulating processes including glycogenolysis in the liver and glycogenesis in the muscles. Epinephrine can be used to treat health problems such as asthma, cardiac arrest, anaphylaxis, and croup. However, it is also known to cause palpitations, tachycardia, arrhythmia, anxiety, panic attacks, hypertension, and acute pulmonary edema.

5. Norepinephrine

Norepinephrine is a neurotransmitter and an adrenal hormone that assists the body in preparing for action. It is at its lowest levels during rest and its highest levels during increased stress or danger. Norepinephrine increases blood pressure in the veins, causing the heart to pump faster. Norepinephrine has been shown to be an effective anti-depressant and is commonly used as such in Europe.

6. Dopamine

Dopamine is another monoamine neurotransmitter that is synthesized in the brain and kidneys. It is often the catalyst for synthesis of norephedrine and ephedrine. Within the nervous system Dopamine instigates rewards, self-motivation, and pleasure. In fact, many addictive drugs often have some interaction with dopamine creating the “high” that subjects feel. Based on the natural effects of dopamine and the many dopamine like affects caused by anti-depressants it is theorized that it plays a major role in depression within the midbrain regions of dopamine neurons.

7. 5-HIAA

5-HIAA, is a metabolite of serotonin and thus is often used to determine the level of serotonin in the body. High levels of 5-HIAA are often used to determine the presence of Carcinoid tumors, Noncarcinoid tumors, Cystic fibrosis, and Malabsorption. Meanwhile low levels can be used to assess or diagnose depression and migraines.

8. Glycine

Glycine is an amino acid and is the most abundant amino acid in collagen. Likewise, it is also an inhibitory neurotransmitter which prevents paralysis in muscle contraction.

9. Histamine

Histamine is a monoamine neurotransmitter which performs many roles within the body. Firstly, histamine lowers blood pressure and causes smooth muscles to relax by releasing other secondary molecules which act as relaxants. These two things can combine to cause anaphylaxis. Histamine release is also connected with many of the symptoms associated with allergies (such as, watery eyes and a runny nose. Histamine release also causes neurons in the hypothalamus to create a feeling of wakefulness, and may suppress convulsions and stress. Histamine is also known to be unbalanced in those with schizophrenia.

10. PEA or Phenethylamine

PEA or Phenethylamine is monoamine that behaves as a stimulant in the central nervous system. It is generally used as a dietary supplement for mood and depression.

11. DOPAC or 3,4-Dihydroxyphenylacetic

DOPAC or 3,4-Dihydroxyphenylacetic acid is a metabolite of dopamine, along with two others (monoamine oxidase [MAO] and catechol-O-methyl transferase [COMT]). Together, they are able to turn dopamine into the end product norepinephrine. DOPAC will occasionally become toxic and destroy dopamine vessels, and is linked to the failure of levodopa in the treatment of Parkinson's Disease.

12. HVA or Homovanillic Acid

HVA or Homovanillic acid is a metabolite and a result of MAO and COMT creation of dopamine. HVA is commonly used to identify tumors (e.g., catecholamine-secreting tumor) as HVA levels decrease due to the tumor.

13. Normetanephrines

Normetanephrine is a metabolite of epinephrine created by COMT. Similar to HVA detection of normetanephrine can be used to suggest the presence of tumors such as Pheochromocytoma. Normetanephrine levels can also be used to identify some adrenalin disorders (through the testing of urine).

14. VMA or Vanillylmandelic Acid

VMA or Vanillylmandelic acid, is the end-stage metabolite of several catecholamines (dopamine, epinephrine, and norepinephrine). VMA levels ca be used to suggest the presence of tumors such as pheochromocytoma.

15. Free Cortisol

Cortisol is a steroid hormone made in the adrenal gland. Cortisol is use for many bodily functions including regulating metabolism, acting as an anti-inflammatory, and controlling water and salt balance. Cortisol is excreted from the adrenal gland during a “flight or fight” response to a perceived fear. Chronically high levels of cortisol can result in vital organ erosion and a variety of mood related problems. There is some association between higher cortisol levels and depression.

### C. Behavioral and Lifestyle Assessments

Within certain aspects of the invention behavioral and lifestyle assessments are conducted as part of the overall diagnosis of a subject, and to provide a basis for therapeutic recommendations. Briefly, the behavioral and lifestyle assessments are surveys that can be used to determine the state of physical, mental, and emotional health of the patient. For each assessment the test taker ranks the degree to which they identify with certain questions that help to qualify.

Within preferred embodiments of the invention the behavioral and/or lifestyle assessments are conducted based upon previously approved and statistically validated questionnaires. Representative examples include: Lehman et al., “Development of the Brief Inventory of Perceived Stress,” J. of Clin. Psych., Vol 68(6), 631-644 (2012); Spitzer et al., “A Brief Measure for Assessing Generalized Anxiety Disorder”, Arch. Intern. Med. Vol 166, May 22, 2006; and Kroenke et al., “The PHQ-9: Validity of a Brief Depression Severity Measure,” J. Gen. Intern. Med., 2001: 16:606-613; all of which are incorporated by reference in their entirety.

As an example, the lifestyle assessment can include, within certain embodiments, questions as to: a) exercise quantity (e.g., the amount of exercise per week); b) exercise intensity (e.g., the intensity or degree of difficulty of exercise per week); c) diet (e.g., the typical dietary intake of the subject); d) amount of sleep (e.g., the quantity in hours the subject sleeps each night); and/or e) quality of sleep (e.g., the quality of sleep each night).

Similarly, a stress inventory can be conducted which determines the level of stress an individual perceives that they face. For example, the subject can be surveyed as to: a) busyness or activity level (e.g., how often the subject feels that they have too many things to do); b) hurriedness (e.g., how often the subject felt they were in a hurry or rushed); c) pressured (e.g., how often they felt that they were under pressure to meet deadlines); d) conflicted (e.g., how often the subject experienced conflict, or, a competitive or antagonistic state); e) choice in matters (e.g., how often the subject felt they had to do things (as opposed to wanted to do things)); f) under examination, review or critique (e.g., how often the subject was subjected to some form of review, examination or criticism); g) pressured (e.g., how often the subject felt that difficulties were piling up); h) falling behind (e.g., how often the subject felt they were not on top of things, or behind on work or projects); and/or i) worrisome (e.g., how often the subject felt that they had too many worries).

The subject can also be queried to assess their level of anxiety. For example, the subject can be asked whether they are: a) nervous or anxious (e.g., feel nervous, anxious or on edge); b) uncontrollable worrying (e.g., feel like they are not able to stop or control worrying, or, worry too much about different things; c) restless (e.g., have trouble relaxing or find it hard to sit still; d) irritable (e.g., are easily annoyed or irritable); and/or g) frightful (e.g., feel afraid as though something bad might happen).

Finally, the subject can also be queried as to their level of depression. For example, the subject can be asked whether they: a) have little interest or pleasure in doing things; b) feel down, depressed or hopeless; c) have trouble falling or staying asleep, or sleeping too much; d) feel tired or have little energy; e) have a poor appetite, or alternatively, overeat; f) feel bad about themselves, or feel a failure; g) moving or speaking slowly, or alternatively, being so restless as to move around more than typical; and/or h) have suicidal thoughts (e.g., have thought that they may better off dead and/or have suicidal thoughts).

Within certain embodiments of the invention any of the aforementioned surveys may be conducted online, through an app (or computer-based application); or other digital means.

### D. Devices, Systems and Methods for Assessing and Diagnosing Mood Disorders

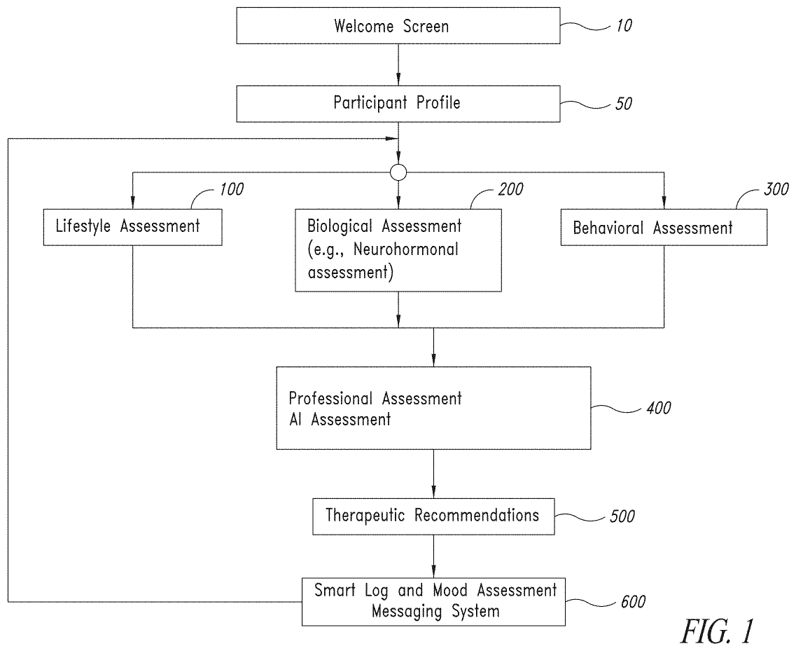

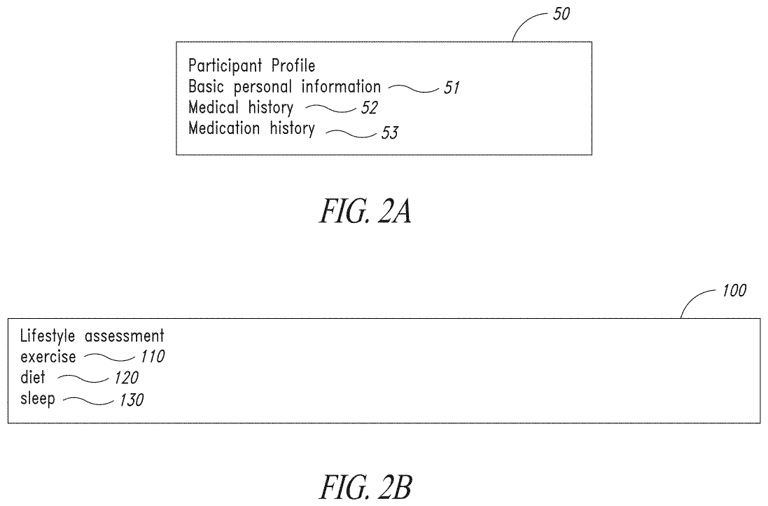

As noted above, the present invention provides a wide variety of devices, systems and methods which are useful for assessing and diagnosing mood disorders. Briefly, within one embodiment of the invention a computer-based system is provided which assists in the diagnosis of an individual with a possible mood disorder. For example, as shown in FIG. 1, an individual can first be introduced to a “Welcome Screen” 10 which orients the individual to a diagnostic program. The individual can then be directed to provide introductory personal information 50 (e.g., as shown in FIG. 2A, personal information 51, medical history 52, medication history 53, etc.); and conduct a number of online surveys (e.g., a lifestyle assessment 100, and a behavioral assessment 300). The subject can also register online for a number of biological marker tests 200 (e.g., neurohormonal tests as discussed in more detail above).

Once all of the testing has been completed, an analysis 400 can be conducted of the resultant information. This analysis can be conducted by a professional (e.g., a physician or qualified therapist). In other embodiments, as described in more detail below, the assessment can also be conducted by using Artificial Intelligence (or “AI”) to conduct the analysis.

The assessment can be used to provide a number of therapeutic recommendations 500. The subject is then tasked with following these therapeutic recommendations, which can be tracked with the Smartlog and ASAP message support system 600 (and as described in more detail below).

This process may, within various embodiments be practiced iteratively to further refine a therapeutic recommendation that works best for an individual subject.

Turning to FIG. 2A, this diagram illustrates an exemplary query for obtaining profile information 50 on a subject. The information can include: a) basic personal information 51; b) a medical history 52; c) medication history 53. FIG. 2B illustrates exemplary queries for a lifestyle assessment 100 (e.g., providing an assessment of exercise 100, diet 120 and/or sleep 130).

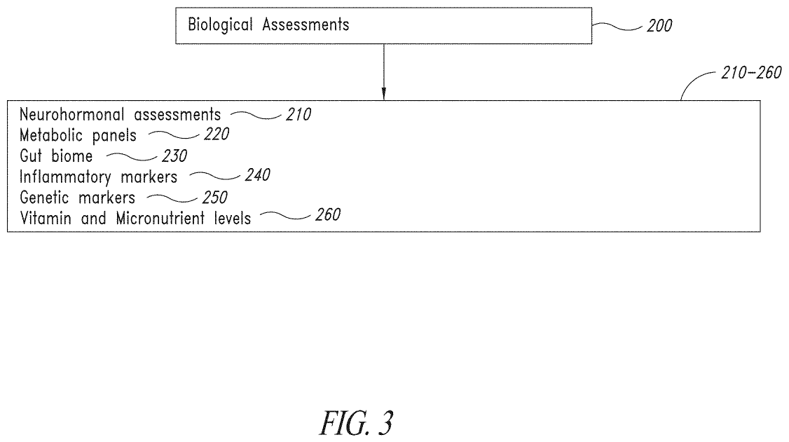

FIG. 3 is a block diagram which illustrates an exemplary method of conducting biological assessments. Within various embodiments of the invention the biological assessment can be one or more of: a) neurohormonal assessments 210; b) metabolic panels 220; c: gut biome analysis 230; d) inflammatory markers 240; e) genetic markers 250; and/or vitamin and/or micronutrient levels 260.

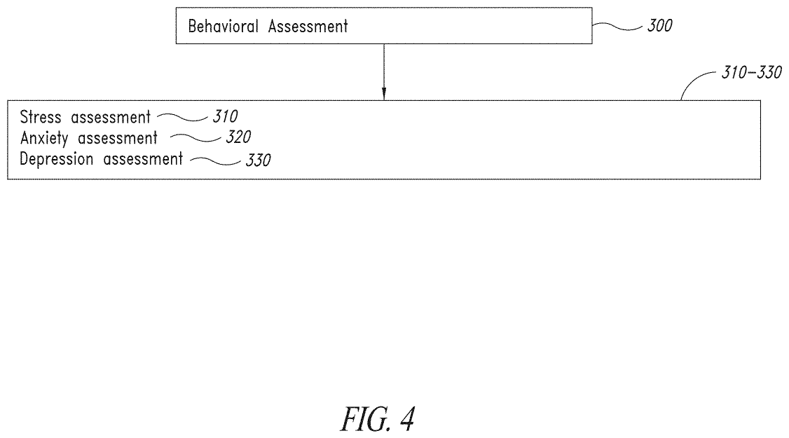

FIG. 4 is a block diagram which illustrates an exemplary method of conducting a behavioral assessment, and illustrative components thereof. Within various embodiments of the invention the lifestyle assessment may include one or more of: a) a stress assessment 310; b) an anxiety assessment 320; and/or c) a depression assessment 330.

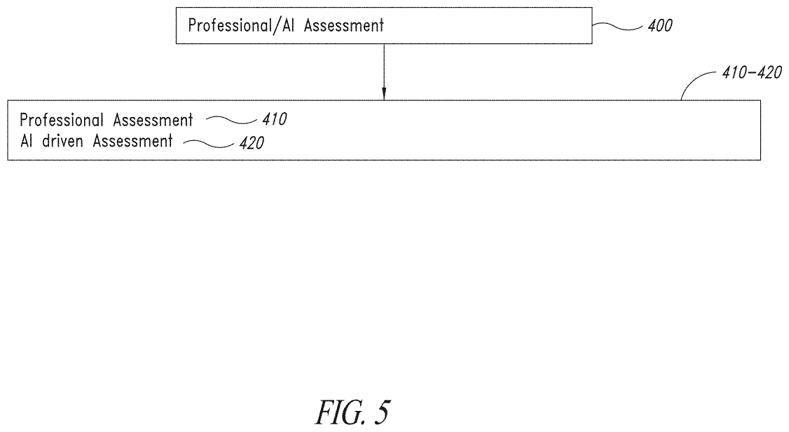

FIG. 5 is a block diagram which illustrates an exemplary method of conducting a professional assessment 410 and/or an AI-driven assessment 420.

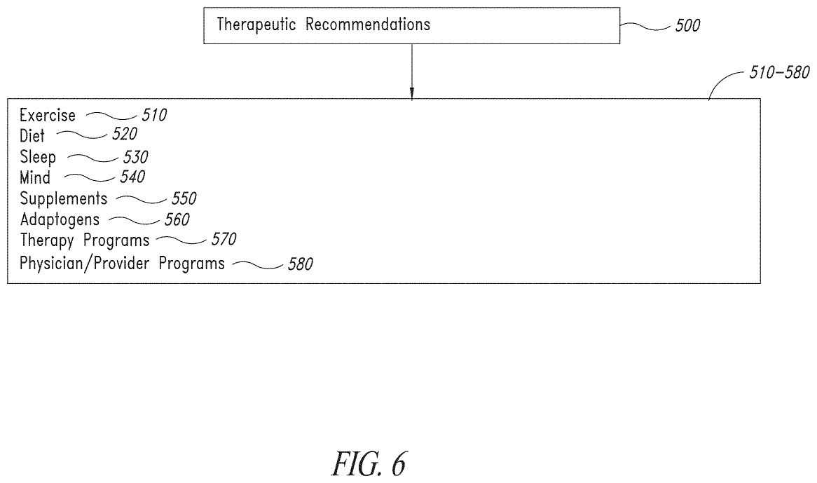

FIG. 6 is a block diagram which illustrates an exemplary method of providing therapeutic recommendations 500. Exemplary therapeutic recommendations can include guidance, goals and or instruction with respect to one or more of: a) exercise 510; b) diet 520; c) sleep 530; d) mind 540; e) supplements 550; f) adaptogens 560; g) therapy programs 570; and/or physician/provider (e.g., therapist) programs 580.

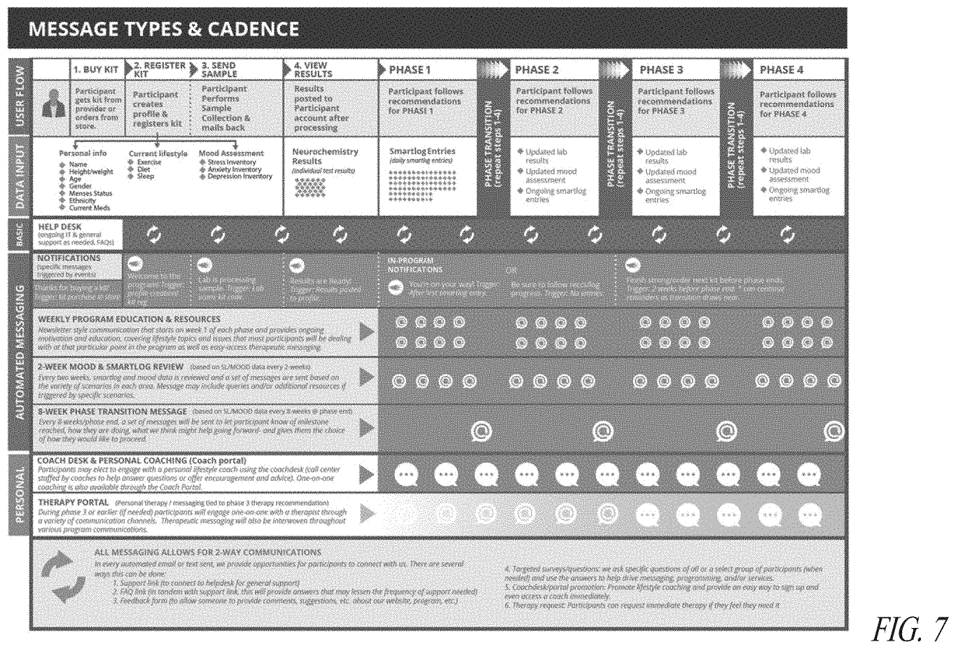

FIG. 7 is a block diagram which illustrates a Smartlog/Messaging system 600 for providing and tracking support. Within certain embodiments of the invention, for each of the therapeutic areas provided above, participant-specific recommendations can be provided. Each set of mood-effective and curative recommendations can be derived from participant-provided profile data (e.g., weight, age, gender, medications, etc.) along with lab provided data (e.g., regarding which neurotransmitters and neurohormones are not at optimal levels). Psychoeducational materials can also be provided to educate participants about the workings of the brain and mind to help them understand how the combination of the two affect their mood status.

The Smartlog is a tool for the assessment of recommendations compliance any one participant receives. The Smartlog monitors and, by its presence, encourages a participant to complete their therapeutic recommendations. When compliance is high then mood levels will be positioned to improve. Within particularly preferred embodiments of the invention a participant can re-assess their mood status every two weeks, or as often as they like, during the program, utilizing the mood assessment suite of three inventories. The Smartlog entries, in combination with the mood assessment suite scores are introduced to the company's AI engine to identify what “types” of participants are more likely to comply and how that compliance has or will affect their mood status and why. Smartlog assessment and ASAP messaging helps the system understand what challenges participants may be facing regarding compliance.

FIG. 7 illustrates one exemplary system of message types and cadence. Briefly, in Phases 1 & 2 where lifestyle assessments are combined with lab results the Subject Matter Experts (“SMEs”) consist of lifestyle (exercise, diet and sleep) experts and laboratory SMEs who analyze urine and saliva-based lab results, factoring in symptomatic elements collected during the onboarding process and medications listed, to provide supplements and adaptogen (supplements that affect brain chemistry) recommendations.

In Phase 3—psychotherapy—the therapist can initially collect a large compliment of participant data from a process referred to as the “intake session” where data on personal history, family history, medical history and raft of other data types determined to have an impact on existing personality traits and behaviors is gathered. When therapy begins detailed session notes, that eventually get translated into “cause and effect” and “diagnostic” data is collected. The therapist can seek to understand the origins/causes of dysfunction and help the participant overcome those dysfunctions through an orchestrated set of “interventions” and “psycho-ed skills training” that will provide the individual with the tools to find relief from their particular mood disorder. This process can take 8 to 12 weeks, in most cases, unless the severity dictates otherwise—in either direction. In Phase 3 the therapist is the SME and their data collection becomes part of the participants total data portrait.

In Phase 4—intervention of a medical doctor (“MD”) for possible medication prescription is considered. All the data previously gathered, including and especially that of the psychotherapist in Phase 3, can be provided in a specialized, summarized format so that the MD can use the data to prescribe the exact medication indicated by all the previous data gathered throughout the phases. The MD in question can select the proper drug classification (antidepressant vs benzodiazepine, for example) and which formulation (manufacturer specific) is the right choice for the participant.

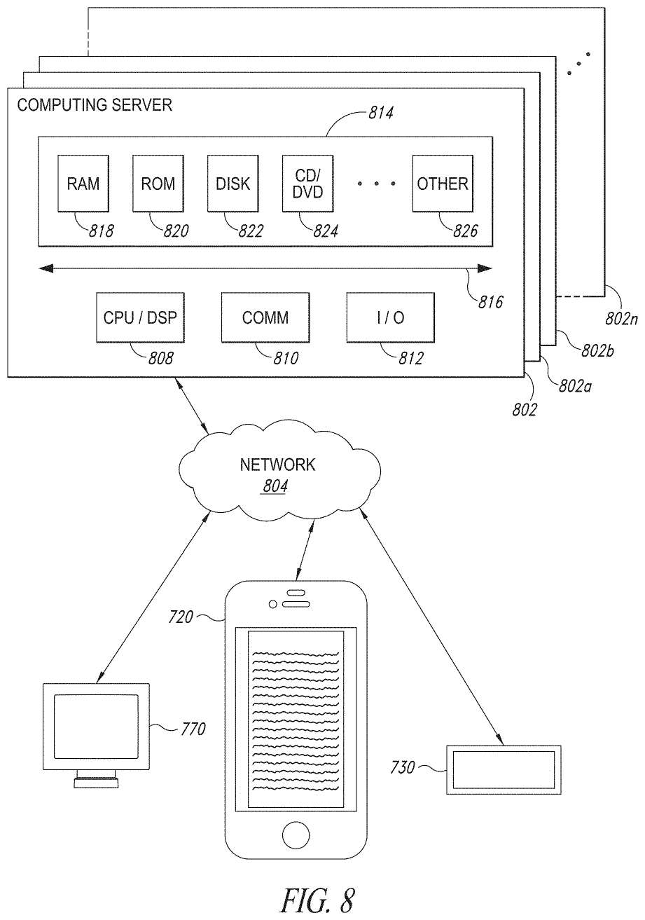

FIG. 8 illustrates one embodiment of an information and communication technology (ICT) system 800 arranged to process data (e.g., data received from a subject on a computer 710, personal communication device such as a phone 720 or watch, and/or from a home communication device or personal assistant such as Amazon's Show 730). In FIG. 8, the ICT system 800 is illustrated to include computing devices that communicate via a network 804, however in other embodiments, the computing devices can communicate directly with each other or through other intervening devices, and in some cases, the computing devices do not communicate at all. The computing devices of FIG. 8 include computing servers 802, and other devices that are not shown for simplicity.

In FIG. 8, one or more sources provide data (e.g., data received from a subject on a computer 710, personal communication device such as a phone 720 or watch, and/or from a home communication device or personal assistant such as Amazon's Show 730) into a network 804. The remote data receiving device can be a wearable device (e.g., a watch-like device, a wristband, or other device that may be carried or worn by the subject). Certain remote devices can collect data on the subject based upon their placement (e.g., a watch could collect heart rate and other biometric data), which can then be forwarded on to one or more networks (804). Alternatively, a remote data receiving device such as a computer 710 or home assistant 730 can be a stationary device in a hospital, home, or office. Representative examples of data that may be collected include location (e.g., a GPS), body or skin temperature, and other physiologic data (e.g., pulse). Within yet other embodiments, the remote data receiving device may notify the subject directly of any of a number of prescribed conditions, including but not limited to possible or actual failure of the device.

The data that is collected (e.g., data received from a subject on a computer 710, personal communication device such as a phone 720 or watch, and/or from a home communication device or personal assistant such as Amazon's Show 730) may be useful for many purposes as described herein. In some cases, for example, data information is collected and analyzed expressly for the health of an individual subject. In other cases, data is collected and transmitted to another computing device to be aggregated with other data.

FIG. 8 illustrates aspects of a computing server 802 as a cooperative bank of servers further including computing servers 802a, 802b, and one or more other servers 802n. It is understood that computing server 802 may include any number of computing servers that operate individually or collectively to the benefit of users of the computing servers.

In some embodiments, the computing servers 802 are arranged as cloud computing devices created in one or more geographic locations, such as the United States and Canada. The cloud computing devices may be created as MICROSOFT AZURE cloud computing devices or as some other virtually accessible remote computing service (e.g., AMAZON's WEB SERVICES (or “AWS”), or MICROSOFT'S AZURE). Within preferred embodiments of the invention, the data is collected and stored in a manner that complies with country specific data privacy laws (e.g., the General Data Protection Requirement or “GDPR” in Europe, ((EU) 2016/679), and the Health Insurance Portability and Accountability Act of 1996 or “HIPAA” in the United States.)

The network 804 includes some or all of cellular communication networks, conventional cable networks, satellite networks, fiber-optic networks, and the like configured as one or more local area networks, wide area networks, personal area networks, and any other type of computing network. In a preferred embodiment, the network 804 includes any communication hardware and software that cooperatively works to permit users of computing devices to view and interact with other computing devices.

Computing server 802 includes a central processing unit (CPU) digital signal processing unit (DSP) 808, communication modules 810, Input/Output (I/O) modules 812, and storage module 814. The components of computing server 802 are cooperatively coupled by one or more buses 816 that facilitate transmission and control of information in and through computing server 802. Communication modules 810 are configurable to pass information between the computer server 802 and other computing devices (e.g., computing servers 802a, 802b, 802n, and the like). I/O modules 812 are configurable to accept input from devices such as keyboards, computer mice, trackballs, and the like. I/O modules 812 are configurable to provide output to devices such as displays, recorders, LEDs, audio devices, and the like.

Storage module 814 may include one or more types of storage media. For example, storage module 814 of FIG. 8 includes random access memory (RAM) 818, read only memory (ROM) 810, disk-based memory 822, optical based memory 824, and other types of memory storage media 8126. In some embodiments one or more memory devices of the storage module 814 has configured thereon one or more database structures. The database structures may be used to store data collected from a variety of devices (e.g., data received from a subject on a computer 710, personal communication device such as a phone 720 or watch, and/or from a home communication device or personal assistant such as Amazon's Show 730), or even data from a sensor or sensors implanted on or within a subject (e.g., a glucose monitor).

In some embodiments, the storage module 814 may further include one or more portions of memory organized a non-transitory computer-readable media (CRM). The CRM is configured to store computing instructions executable by a CPU 808. The computing instructions may be stored as one or more files, and each file may include one or more computer programs. A computer program can be standalone program or part of a larger computer program. Alternatively or in addition, each file may include data or other computational support material for an application that directs the collection, analysis, processing, and/or distribution of data. The data application typically executes a set of instructions stored on computer-readable media.

It will be appreciated that the computing servers shown in the figures and described herein are merely illustrative and are not intended to limit the scope of the present invention. Computing server 802 may be connected to other devices that are not illustrated, including through one or more networks such as the Internet or via the Web that are incorporated into network 804. More generally, a computing system or device (e.g., a “client” or “server”) or any part thereof may comprise any combination of hardware that can interact and perform the described types of functionality, optionally when programmed or otherwise configured with software, including without limitation desktop or other computers, database servers, network storage devices and other network devices, PDAs, cell phones, glasses, wrist bands, wireless phones, pagers, electronic organizers, Internet appliances, television-based systems (e.g., using set-top boxes and/or personal/digital video recorders), and various other products that include appropriate inter-communication capabilities. In addition, the functionality provided by the illustrated system modules may in some embodiments be combined in fewer modules or distributed in additional modules. Similarly, in some embodiments the functionality of some of the illustrated modules may not be provided and/or other additional functionality may be available.

In addition, while various items are illustrated as being stored in memory or on storage while being used, these items or portions of them can be transferred between memory and other storage devices for purposes of memory management and/or data integrity. In at least some embodiments, the illustrated modules and/or systems are software modules/systems that include software instructions which, when executed by the CPU/DSP 808 or other processor, will program the processor to automatically perform the described operations for a module/system. Alternatively, in other embodiments, some or all of the software modules and/or systems may execute in memory on another device and communicate with the illustrated computing system/device via inter-computer communication.

Furthermore, in some embodiments, some or all of the modules and/or systems may be implemented or provided in other manners, such as at least partially in firmware and/or hardware means, including, but not limited to, one or more application-specific integrated circuits (ASICs), standard integrated circuits, controllers (e.g., by executing appropriate instructions, and including microcontrollers and/or embedded controllers), field-programmable gate arrays (FPGAs), complex programmable logic devices (CPLDs), and the like. Some or all of the systems, modules, or data structures may also be stored (e.g., as software instructions or structured data) on a transitory or non-transitory computer-readable storage medium 814, such as a hard disk 822 or flash drive or other non-volatile storage device 826, volatile 818 or non-volatile memory 810, a network storage device, or a portable media article (e.g., a DVD disk, a CD disk, an optical disk, a flash memory device, etc.) to be read by an appropriate input or output system or via an appropriate connection. The systems, modules, and data structures may also in some embodiments be transmitted as generated data signals (e.g., as part of a carrier wave or other analog or digital propagated signal) on a variety of computer readable transmission mediums, including wireless-based and wired/cable-based mediums. The data signals can take a variety of forms such as part of a single or multiplexed analog signal, as multiple discrete digital packets or frames, as a discrete or streaming set of digital bits, or in some other form. Such computer program products may also take other forms in other embodiments. Accordingly, the present invention may be practiced with other computer system configurations. Representative examples, include those that are described in U.S. Pat. Nos. 8,301,467, 8,679,009, 10,172,550, 10,204,707, and 10,687,751, all of which are incorporated by reference in their entirety.

In FIG. 8, data (e.g., data received from a subject on a computer 770, personal communication device such as a phone 720 or watch, and/or from a home communication device or personal assistant such as Amazon's Show 730) is provided to computing server 802. Generally speaking, the data, represents data retrieved from a known subject. The data may possess include or be further associated with additional information such a time stamp, a location (e.g., GPS) stamp, a date stamp, and other information.

In some embodiments, the data may comprise sensitive information such as private health information associated with a specific subject. Sensitive information may include any information that an associated party desires to keep from wide or easy dissemination. Sensitive information can stand alone or be combined with other non-sensitive information. For example, a subject's medical information is typically sensitive information. In some cases, the storage and transmission of a subject's medical information is protected by a government directive (e.g., law, regulation, etc.) such as the U.S. Health Insurance Portability and Accountability Act (HIPAA).

As discussed herein, a reference to “sensitive” information includes information that is entirely sensitive and information that is some combination of sensitive and non-sensitive information. The sensitive information may be represented in a data file or in some other format. As used herein, a data file that includes a subject's medical information may be referred to as “sensitive information.” Other information, such as employment information, financial information, identity information, and many other types of information may also be considered sensitive information.

A computing system can represent sensitive information with an encoding algorithm (e.g., ASCII), a well-recognized file format (e.g., PDF), or by some other format. In a computing system, sensitive information can be protected from wide or easy dissemination with an encryption algorithm.

Generally speaking, sensitive information can be stored by a computing system as a discrete set of data bits. The set of data bits may be called “plaintext.” Furthermore, a computing system can use an encryption process to transform plaintext using an encryption algorithm (i.e., a cipher) into a set of data bits having a highly unreadable state (i.e., cipher text). A computing system having knowledge of the encryption key used to create the cipher text can restore the information to a plaintext readable state. Accordingly, in some cases, sensitive data (e.g., data 806a, 806b) is optionally encrypted before being communicated to a computing device.

In one embodiment, the operation of the information and communication technology (ICT) system 800 of FIG. 8 includes one or more data computer programs stored on a computer-readable medium. The computer program may optionally direct and/or receive data from one or more sources in one or more subjects. A data computer program may be executed in a computing server 802. Alternatively, or in addition, a data computer program may be executed in a control unit 126, an interrogation unit 124.

Within certain embodiments of the invention, the data obtained from the subject (e.g., profile information, biological assessments, and/or behavioral assessments) are analyzed using computer-generated artificial intelligence. Briefly, within certain embodiments of the invention computer-generated artificial intelligence allows a computing system to analyze multiple different factors, and to, either directly or by comparison to other know subjects and/or data sources, derive specific therapeutic recommendations. Representative examples of computer-generated artificial intelligence programs are described in more detail in US Patent Application Nos. US20190228297; US20190187785; US20180246883; US20170299802 and U.S. Pat. Nos. 4,670,848; 6,738,753; and 8,175,981, all of which are incorporated by reference in their entirety. Commercially available AI platforms are available from Microsoft AZURE AI; Amazon Web Services Artificial Intelligence and Machine Learning; Google AI; SAS Data and Analytics; and IBM's Artificial Intelligence WATSON.

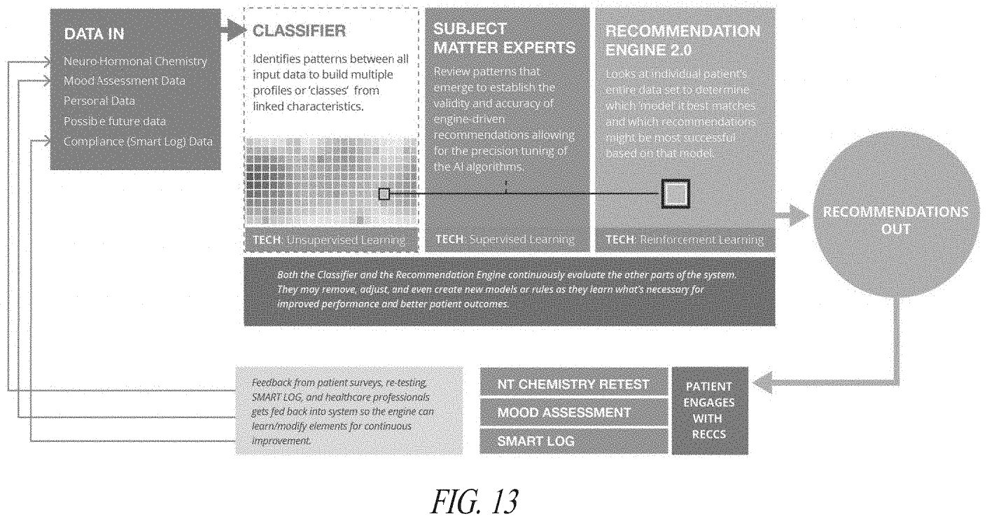

FIG. 13 diagrammatically illustrates one embodiment of a recommendation system which can employ subject matter experts and artificial intelligence or machine learning. For example: in a method; or, in a non-transitory computer-readable storage medium whose stored contents configure a computing system to perform a method; or in a computing system, comprising one or more processors; memory; and one or more programs, wherein the one or more programs are stored in the memory and are configured to be executed by the one or more processors, to diagnose a first subject of a mood disorder and to provide treatment recommendations for the first subject, the following methods as generally set forth in FIG. 13 can be accomplished. Briefly, (1) a variety of information is provided into one or more databases, wherein the information includes (a) personal information obtained from subject; (b) a lifestyle assessment of said subject; (c) a biological assessment of said subject (e.g., neurohormonal chemistry), and (d) a behavioral assessment of said subject. Optionally, further data may be included with respect to previous rounds of testing of the subject, as well as the results of any therapeutic recommendations. (2) This information is then classified; specifically, patterns between multiple individuals are defined in order to identify profiles or ‘classes’ with linked characteristics. The information may be classified by subject matter experts and/or by one or more artificial intelligence or machine learning algorithms, in order to identify patterns that emerge, and to establish the validity and accuracy of the classifications. (3) A ‘recommendation engine’ (e.g., one or more artificial intelligence or machine learning algorithms) can be used to look at individual subjects within the entire data set to determine which therapeutic recommendation might suit a particular subject, based upon the multiple individuals that have been included in the database. Within certain embodiments of the invention at least 1,000, at least 5,000, 10,000, 50,000, or, greater than 100,000 individuals are profiled in said one or more databases, allowing for a better therapeutic recommendation for said subject to be generated.

Increasingly more precise therapeutic recommendations for a subject and more importantly, for similarly situated subjects to follow depend upon and are a function of the feedback of the outcome data generated by subjects engaging in the therapeutic recommendations. In this way, continual capture of behavioral assessments drives the reinforcement-based machine learning algorithms to achieve greater levels of quality therapeutic recommendations.

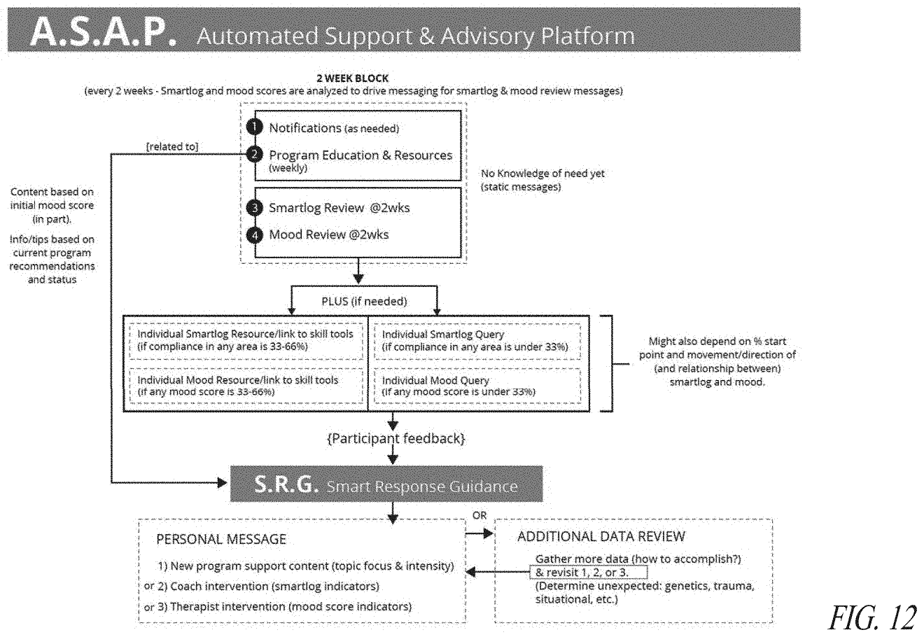

The above method can be followed up to determine how well the patient complied with the therapeutic recommendations, through (a) a Smart Log (as described, e.g., in FIG. 12); (b) further lifestyle assessments; (c) further biological assessments; and/or (d) further behavioral assessments.

Those skilled in the art will recognize that it is common within the art to implement devices and/or processes and/or systems, and thereafter use engineering and/or other practices to integrate such implemented devices and/or processes and/or systems into more comprehensive devices and/or processes and/or systems. That is, at least a portion of the devices and/or processes and/or systems described herein can be integrated into other devices and/or processes and/or systems via a reasonable amount of experimentation. Those having skill in the art will recognize that examples of such other devices and/or processes and/or systems might include—as appropriate to context and application—all or part of devices and/or processes and/or systems of (a) an air conveyance (e.g., an airplane, rocket, helicopter, etc.), (b) a ground conveyance (e.g., a car, ambulance, truck, locomotive, tank, armored personnel carrier, etc.), (c) a building (e.g., a home, hospital, warehouse, office, etc.), (d) an appliance (e.g., a refrigerator, a washing machine, a dryer, etc.), (e) a communications system (e.g., a networked system, a telephone system, a Voice over IP system, etc.), (f) a business entity (e.g., an Internet Service Provider (ISP) entity such as Comcast Cable, Qwest, Southwestern Bell, etc.), or (g) a wired/wireless services entity (e.g., AT&T, T-Mobile, Verizon.), etc.

In certain cases, use of a system or method may occur in a territory even if components are located outside the territory. For example, in a distributed computing context, use of a distributed computing system may occur in a territory even though parts of the system may be located outside of the territory (e.g., relay, server, processor, signal-bearing medium, transmitting computer, receiving computer, etc. located outside the territory).

A sale of a system or method may likewise occur in a territory even if components of the system or method are located and/or used outside the territory. Further, implementation of at least part of a system for performing a method in one territory does not preclude use of the system in another territory.

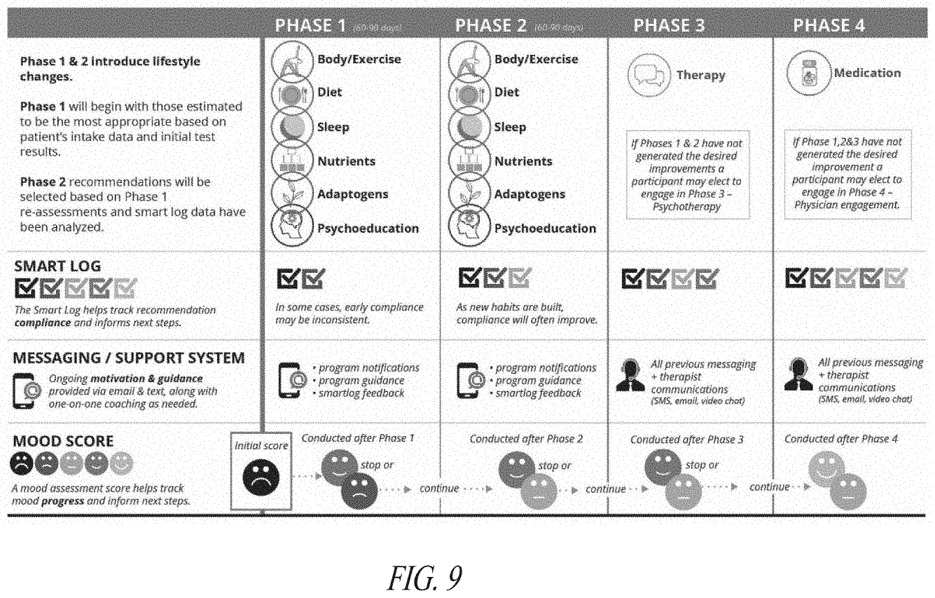

FIG. 9 diagrammatically illustrates one embodiment of various phases of treatment and possible mood status responses thereto;

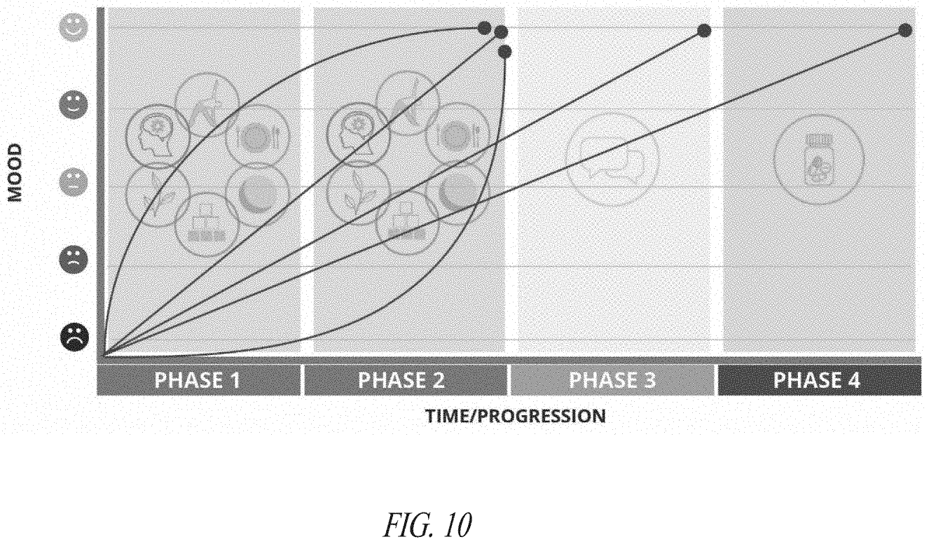

FIG. 10 diagrammatically illustrates the time/progression of a subject's possible mood status progression during the course of multiple phases of diagnosis and treatment.

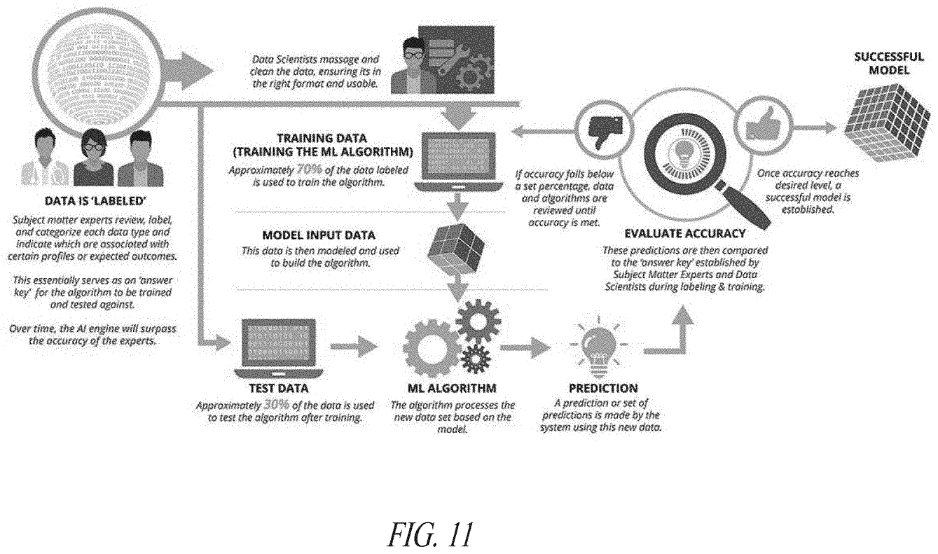

FIG. 11 diagrammatically illustrates one embodiment of data evaluation as it pertains to how the AI engine is trained with program-assembled and analyzed data.

FIG. 12 diagrammatically illustrates one embodiment of an automated support and advisory system.

### Conventions

In general, and unless otherwise specified, all technical and scientific terms used herein shall have the same meaning as those commonly understood by one of ordinary skill in the art to which the embodiment pertains. For convenience, the meanings of selected terms are provided below, where these meanings are provided in order to aid in describing embodiments identified herein. Unless stated otherwise, or unless implicit from the context in which the term is used, the meanings provided below are the meanings intended for the referenced term.

Embodiment examples or feature examples specifically provided are intended to be exemplary only, that is, those examples are non-limiting on an embodiment. The term “e.g.” (Latin, exempli gratia) is used herein to refer to a non-limiting example, and effectively means “for example”. In addition, the Figures, while being understood to generally show the subject matter being described, should not be seen as limiting.

Except in specific examples provided herein, or where otherwise indicated, all numbers expressing quantities of a component should be understood as modified in all instances by the term “about”, where “about” means ±5% of the stated value, e.g., 100 refers to any value within the range of 95-105.

The invention has been described broadly and generically herein. Each of the narrower species and sub generic groupings falling within the generic disclosure also form part of the invention. This includes the generic description of the invention with a proviso or negative limitation removing any subject matter from the genus, regardless of whether or not the excised material is specifically recited herein.

The following are some exemplary numbered embodiments of the present disclosure.

1. A method of assessing mood disorder within a subject, comprising the steps of
(a) obtaining personal information from said subject;
(b) conducting a lifestyle assessment;
(c) conducting a biological assessment; and
(d) conducting a behavioral assessment.
   Within various embodiments of the invention the method further comprises the step of developing therapeutic recommendations for treating a subject.

2. The method of embodiments 1 or 2, wherein said personal information includes one or more of (a) the subject's name; (b) an address; (c) a phone number; (d) gender of the subject; and/or (e) social identification number or other subject specific identification sequence.

3. The method of embodiments 1, 2, or 4, wherein the lifestyle assessment measures the exercise, sleep and/or diet habits of a subject. Within certain embodiments of the invention, the lifestyle assessment appraises (and optionally quantifies) a subject's exercise quantity and intensity, diet, and/or quantity and quality of sleep.

4. The method of any one of embodiments 1 to 3, wherein the biological assessment is a neuro-hormonal assessment. Within various embodiments of the invention, the neuro-hormonal assessment measures the presence, level or quantity of one or more of: serotonin, dopamine, GABA, Glutamate, PEA, norepinephrine, epinephrine, cortisol, DNEA, estrone, Estradiol, Estriol, progesterone, and/or testosterone. Within other embodiments the neurohormonal assessment integrates values from both brain and body chemistry, namely, specific brain chemistry in concert with adrenal-produced hormones/neurohormones (those directly affecting brain chemistry)

5. The method of any one of embodiments 1 to 4 wherein said behavioral assessment determines the stress, anxiety and/or depression levels of a subject. Within certain embodiments of the invention, the behavioral assessment appraises (and optionally quantifies) a subject's perceived level of stress (e.g., feelings of busyness or hurriedness, pressure, conflict and/or worries). Within other embodiments of the invention, the behavioral assessment appraises (and optionally quantifies) a subject's perceived level of anxiety (e.g., feelings of nervousness, uncontrollable worrying, restlessness and/or frightfulness). Within yet other embodiments of the invention, the behavioral assessment appraises (and optionally quantifies) a subject's perceived level of depression (including for example, prevalence and/or degree of hopelessness and/or suicidal thoughts).

6. The method of any one of embodiments 1 to 5, further comprising the step of developing a therapeutic recommendation from said personal information, lifestyle assessment, biological assessment, and/or behavioral assessment. Within certain embodiments of the invention this recommendation is performed by a human subject matter expert.

7. The method of any one of embodiments 1 to 6, further comprising the step of using computer-generated artificial intelligence to analyze said information and assessments and develop a therapeutic recommendation. Within certain embodiments Subject Matter Experts (SMEs) supervise the training of the AI engine to improve output accuracy with each new subject leading to precision analysis and assessments in developing therapeutic treatment recommendations.

8. The method of any one of embodiments 1 to 7 wherein said personal information, lifestyle assessment and behavioral assessment is obtained from a single online questionnaire.

9. The method of any one of embodiments 1 to 8 wherein said subject submits an online request to receive a biological assessment kit. Within further embodiments, the biological assessment is performed by a third-party laboratory.

10. The method of any one of embodiments 1 to 9 wherein said recommendation is developed by comparing a subject's assessments against a database of a plurality of second subject's assessments, wherein said second subject's assessments also include datasets of: (a) the lifestyle, biological assessment and behavioral assessments of said second subjects; (b) therapeutic regimens of said second subjects; (c) compliance of said second subjects with said therapeutic regimen; and/or (d) success of a particular therapeutic regimen for a second subject.

Within various embodiments of the above, the method may further comprise the step of monitoring the subject to assess compliance with the therapeutic recommendation. Within yet other embodiments the method may also further comprise the step of reinforcing a subject's compliance with the therapeutic recommendation.

Within further aspects of the present invention, any of the above embodiments can be repeated multiple time (e.g., 1, 2, 3, 4 or 5 times) in order to generate a more successful therapeutic recommendation for the subject. If, alternatively, after multiple repeated attempts (e.g., 1, 2, 3, or 4 times) a suitable or successful therapeutic recommendation cannot be generated for the subject, the subject can be referred to a physician for treatment with prescription medicines.

11. A non-transitory computer-readable storage medium whose stored contents configure a computing system to perform a method, the method comprising:
(a) maintaining at least one database having information stored therein, said database being organized as a plurality of records, wherein each of said records has a plurality of fields;
(b) providing information to said database, to be stored in one of said records, wherein said information comprises subject specific personal information, lifestyle information, behavioral information, and/or biological assessment information which is specific to said subject; and
(c) analyzing said subject specific personal information to determine a therapeutic recommendation. Within certain embodiments of the invention the therapeutic recommendation can be generated by computer-generated artificial intelligence which analyzes said information and assessments and develop a therapeutic recommendation.

12. The non-transitory computer-readable storage medium according to embodiment 11, wherein said subject specific personal information includes one or more of (a) the subject's name; (b) an address; (c) a phone number; (d) gender of the subject; and/or (e) social identification number or other subject specific identification sequence.

13. The non-transitory computer-readable storage medium according to any one of embodiments 11 to 12, wherein said lifestyle information includes the exercise, sleep and/or diet habits of a subject.

14. The non-transitory computer-readable storage medium according to any one of embodiments 1 to 12, wherein said behavioral information includes the stress, anxiety and/or depression levels of a subject.

15. The non-transitory computer-readable storage medium according to any one of embodiments 1 to 14, wherein said biological assessment information which is specific to said subject includes a neurohormonal assessment.

16. A storage medium according to any one of embodiments 11 to 15, wherein said subject specific personal information is collected from said subject online. Within other embodiments, the subject specific personal information may be collected by a health care provider.

17. A storage medium according to embodiment 16 wherein said lifestyle information and behavioral information, is collected from a subject online. Within other embodiments, the lifestyle information and/or behavioral information may be collected by a health care provider.

18. A storage medium according to embodiments 16 or 17 wherein said biological marker information is collected from a laboratory.

19. The storage medium according to any one of embodiments 16 to 18 wherein all or a part of said information is provided to a health care provider.

20. A computing system, comprising: one or more processors; memory; and one or more programs, wherein the one or more programs are stored in the memory and are configured to be executed by the one or more processors to diagnose a first subject of a mood disorder and to provide treatment recommendations for said subject, the one or more programs including instructions for: a) obtaining subject information; b) obtaining a lifestyle assessment from said subject, c) obtaining biological assessment information on said subject (optionally through a third party laboratory); and d) obtaining behavioral assessment information.

21. The computing system of embodiment 20, further comprising the step of generating an assessment of said first subject. Within certain embodiments of the invention, the assessment can be generated by comparing the information from said first subject with a database comprising a plurality of second subjects.

22. The computing system of embodiments 20 or 21, further comprising the step of generating a therapeutic recommendation for said first subject. Within certain embodiments of the invention, the therapeutic recommendation can be generated by comparing information obtained from said first subject with information collected from a plurality of second subjects, and analyzing successfully treated second subjects to generate a therapeutic recommendation for said first subject. Within further embodiments, the therapeutic recommendation is generated using artificial intelligence or machine learning.

23. The computing system according to any one of embodiments 20 to 22, further comprising the step of monitoring implementation and compliance of the therapeutic recommendations.

24. The computing system according to any one of embodiments 20 to 23, further comprising repeating the steps of any one of embodiments 1 to 11. Within certain embodiments, embodiment 1(b), 1(c), 1(d), 2, and/or 3 can be repeated cycle, in order to further refine a therapeutic recommendation.

25. The computing system according to any one of embodiments 20 to 24, wherein the therapeutic recommendation reduces the incidence or severity of mood disorders in each cycle.

26. Within further aspects of any of embodiments 1 to 25, the subject can obtain a therapeutically effective outcome. Within certain embodiments, the incidence or severity one or more negative factors (e.g., a negative lifestyle assessment, a negative biological assessment, and/or a negative behavioral assessment will be decreased by at least 10%, 20%, 25%, 50%, or greater than 75% in each cycle. For example, the level of stress, depression or anxiety will be decreased in a statistically meaningful manner for a subject that has completed one, and more preferably, multiple rounds of the assessment and recommendation cycles described herein. Within further embodiments of the invention, each recommendation cycle can include an assessment to determine the percentage decrease in negative factors influencing a subject's mood status as reflected in the direct assessment of the individual's mood levels. Such assessments represent the outcome, or therapeutic results, of each set of recommendations. Increasingly precise therapeutic recommendations for a subject to follow, and more importantly, for similarly related subjects will depend upon and are a function of the feedback of the outcome data generated by subjects engaging in the therapeutic recommendations. Behavioral outcome data captured in this manner (a unique feature of this invention), drive the reinforcement-based machine learning algorithms to achieve greater levels of predictive excellence relative to the therapeutic recommendations.

It should be understood that as used herein and in the appended claims, the singular forms “a,” “an,” and “the” include plural reference unless the context clearly dictates otherwise, the term “X and/or Y” means “X” or “Y” or both “X” and “Y”, and the letter “s” following a noun designates both the plural and singular forms of that noun. In addition, where features or aspects of the invention are described in terms of Markush groups, it is intended, and those skilled in the art will recognize, that the invention embraces and is also thereby described in terms of any individual member and any subgroup of members of the Markush group, and Applicants reserve the right to revise the application or claims to refer specifically to any individual member or any subgroup of members of the Markush group.

It is to be understood that the terminology used herein is for the purpose of describing specific embodiments only and is not intended to be limiting. It is further to be understood that unless specifically defined herein, the terminology used herein is to be given its traditional meaning as known in the relevant art.

Reference throughout this specification to “one embodiment” or “an embodiment” and variations thereof means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents, i.e., one or more, unless the content and context clearly dictates otherwise. It should also be noted that the conjunctive terms, “and” and “or” are generally employed in the broadest sense to include “and/or” unless the content and context clearly dictates inclusivity or exclusivity as the case may be. Thus, the use of the alternative (e.g., “or”) should be understood to mean either one, both, or any combination thereof of the alternatives. In addition, the composition of “and” and “or” when recited herein as “and/or” is intended to encompass an embodiment that includes all of the associated items or ideas and one or more other alternative embodiments that include fewer than all of the associated items or ideas.

Unless the context requires otherwise, throughout the specification and claims that follow, the word “comprise” and synonyms and variants thereof such as “have” and “include”, as well as variations thereof such as “comprises” and “comprising” are to be construed in an open, inclusive sense, e.g., “including, but not limited to.” The term “consisting essentially of” limits the scope of a claim to the specified materials or steps, or to those that do not materially affect the basic and novel characteristics of the claimed invention.

Any headings used within this document are only being utilized to expedite its review by the reader and should not be construed as limiting the invention or claims in any manner. Thus, the headings and Abstract of the Disclosure provided herein are for convenience only and do not interpret the scope or meaning of the embodiments.

Where a range of values is provided herein, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

For example, any percentage range, ratio range, or integer range provided herein is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated.

All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Such documents may be incorporated by reference for the purpose of describing and disclosing, for example, materials and methodologies described in the publications, which might be used in connection with the presently described invention. The publications discussed above and throughout the text are provided solely for their disclosure prior to the filing date of the present application. Nothing herein is to be construed as an admission that the inventors are not entitled to antedate any referenced publication by virtue of prior invention.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

Furthermore, the written description portion of this patent includes all claims. Furthermore, all claims, including all original claims as well as all claims from any and all priority documents, are hereby incorporated by reference in their entirety into the written description portion of the specification, and Applicants reserve the right to physically incorporate into the written description or any other portion of the application, any and all such claims. Thus, for example, under no circumstances may the patent be interpreted as allegedly not providing a written description for a claim on the assertion that the precise wording of the claim is not set forth in haec verba in written description portion of the patent.

The claims will be interpreted according to law. However, and notwithstanding the alleged or perceived ease or difficulty of interpreting any claim or portion thereof, under no circumstances may any adjustment or amendment of a claim or any portion thereof during prosecution of the application or applications leading to this patent be interpreted as having forfeited any right to any and all equivalents thereof that do not form a part of the prior art.

Other nonlimiting embodiments are within the following claims. The patent may not be interpreted to be limited to the specific examples or nonlimiting embodiments or methods specifically and/or expressly disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

What is claimed is:

1. A method of assessing mood disorder within a subject, comprising the steps of
(a) obtaining personal information from said subject;
(b) conducting a lifestyle assessment;
(c) conducting a biological assessment; and
(d) conducting a behavioral assessment.

2. The method of claim 1, wherein said personal information includes one or more of (a) the subject's name; (b) an address; (c) a phone number; (d) gender of the subject; and/or
(e) social identification number or other subject specific identification sequence.

3. The method of claim 1, wherein the lifestyle assessment measures the exercise, sleep and/or diet habits of a subject.

4. The method of claim 1, wherein the biological assessment is a neuro-hormonal assessment.

5. The method of claim 1 wherein said behavioral assessment determines the stress, anxiety and/or depression levels of a subject.

6. The method of claim 1, further comprising the step of developing a therapeutic recommendation from said personal information, lifestyle assessment, biological assessment, and/or behavioral assessment.

7. The method of claim 5, further comprising the step of using computer-generated artificial intelligence to analyze said information and assessments and develop a therapeutic recommendation.

8. The method of claim 1, wherein said personal information, lifestyle assessment and behavioral assessment is obtained from a single online questionnaire.

9. The method of claim 1 wherein said subject submits an online request to receive a biological assessment kit.

10. The method of claim 5, wherein said recommendation is developed by comparing a subject's assessments against a database of a plurality of second subject's assessments, wherein said second subject's assessments also include datasets of: (a) the lifestyle, biological assessment and behavioral assessments of said second subjects; (b) therapeutic regimens of said second subjects; (c) compliance of said second subjects with said therapeutic regimen; and/or (d) success of a particular therapeutic regimen for a second subject.

11. A non-transitory computer-readable storage medium whose stored contents configure a computing system to perform a method, the method comprising:
(d) maintaining at least one database having information stored therein, said database being organized as a plurality of records, wherein each of said records has a plurality of fields;
(e) providing information to said database, to be stored in one of said records, wherein said information comprises subject specific personal information, lifestyle information, behavioral information, and/or biological assessment information which is specific to said subject; and
(f) analyzing said subject specific personal information to determine a therapeutic recommendation.

12. The non-transitory computer-readable storage medium according to claim 10, wherein said subject specific personal information includes one or more of (a) the subject's name; (b) an address; (c) a phone number; (d) gender of the subject; and/or (e) social identification number or other subject specific identification sequence.

13. The non-transitory computer-readable storage medium according to claim 10, wherein said lifestyle information includes the exercise, sleep and/or diet habits of a subject.

14. The non-transitory computer-readable storage medium according to claim 10, wherein said behavioral information includes the stress, anxiety and/or depression levels of a subject.

15. The non-transitory computer-readable storage medium according to claim 10, wherein said biological assessment information which is specific to said subject includes a neurohormonal assessment.

16. A storage medium according to any one of claims 11 to 15, wherein said subject specific personal information is collected from said subject online.

17. A storage medium according to any one of claims 11 to 16 wherein said lifestyle information and behavioral information, is collected from a subject online.

18. A storage medium according to any one of claims 11 to 17 wherein said biological marker information is collected from a laboratory.

19. The storage medium according to any one of claims 11 to 18 wherein all or a part of said information is provided to a health care provider.

20. A computing system, comprising: one or more processors; memory; and one or more programs, wherein the one or more programs are stored in the memory and are configured to be executed by the one or more processors to diagnose a first subject of a mood disorder and to provide treatment recommendations for said subject, the one or more programs including instructions for: a) obtaining subject information; b) obtaining a lifestyle assessment from said subject, c) obtaining biological assessment information on said subject (optionally through a third party laboratory); and d) obtaining behavioral assessment information.
